(19) United States
(12) Patent Application Publication
(10) Pub. No.: US 2024/0358507 A1
(43) Pub. Date: Oct. 31, 2024

(54) STABILIZED IMPLANT DEPLOYMENT SYSTEM

(71) Applicant: Medtronic, Inc, Minneapolis, MN (US)

(72) Inventors: Erik C. Griswold, Penngrove, CA (US); Austin G, Haynes, Santa Rosa, CA (US); Amanda L. Fazekas, San Diego, CA (US); Cian M. Ryan, Santa Rosa, CA (US)

(21) Appl. No.: 18/566,049

(22) PCT Filed: May 31, 2022
(86) PCT No.: PCT/IB2022/055090

§ 371 Date: Nov. 30, 2023

Related U.S. Application Data
(60) Provisional application No. 63/197,048, filed on Jun. 4, 2021.

(51) Int. Cl. A61F 2/24; A61F 2/958

(52) U.S. Cl. CPC A61F 2/2433; A61F 2/2418; A61F 2/2436; A61F 2002/9583; A61F 2230/0069

(57) ABSTRACT

Aspects of the disclosure relate to systems, delivery devices and method for transcatheter delivery of a cardiac implant such as a prosthetic heart valve. Systems of the disclosure are configured to support the delivery device in an inferior vena cava and/or superior vena cava to reduce the potential for damage to heart tissue during delivery of the implant as the delivery device is articulated through the vasculature.

### FIELD

The present technology is generally related to systems for stabilizing an implant delivery device for treatment of a tricuspid or mitral heart valve, for example.

### BACKGROUND

A human heart includes four heart valves that determine the pathway of blood flow through the heart: the mitral valve, the tricuspid valve, the aortic valve, and the pulmonary valve. The mitral and tricuspid valves are atrioventricular valves, which are between the atria and the ventricles, while the aortic and pulmonary valves are semilunar valves, which are in the arteries leaving the heart. Ideally, native leaflets of a heart valve move apart from each other when the valve is in an open position, and meet or “coapt” when the valve is in a closed position. Problems that may develop with valves include stenosis in which a valve does not open properly, and/or insufficiency or regurgitation in which a valve does not close properly. Stenosis and insufficiency may occur concomitantly in the same valve. The effects of valvular dysfunction vary, with regurgitation or backflow typically having relatively severe physiological consequences to the patient.

Recently, flexible prosthetic valves supported by stent structures that can be delivered percutaneously using a catheter-based delivery system have been developed for heart and venous valve replacement. These prosthetic valves may include either self-expanding or balloon-expandable stent structures with valve leaflets attached to the interior of the stent structure. The prosthetic valve can be reduced in diameter, by crimping onto a balloon catheter or by being contained within a sheath component of a delivery catheter, and advanced through the venous or arterial vasculature. Once the prosthetic valve is positioned at the treatment site, for instance within an incompetent native valve, the stent structure may be expanded to hold the prosthetic valve firmly in place.

The present disclosure addresses problems and limitations associated with the related art.

### SUMMARY

The techniques of this disclosure generally relate to systems and methods of navigating and control of a delivery device maintaining a cardiac implant while supporting and stabilizing of the delivery device to allow for precise and stable positioning of the implant within a heart valve, such as a tricuspid or mitral heart valve.

In one aspect, the present disclosure provides a system including a delivery device having an outer catheter and an inner catheter supporting an implant. The system further includes a stabilization device positioned at least partially over the outer catheter. The stabilization device including a distal end and a proximal end. The stabilization device including an outer surface and an opening within the outer surface positioned between the distal end and the proximal end. The system includes an arrangement in which the outer catheter at least partially extends out of the opening.

In another aspect, the disclosure provides a system including a delivery device having an outer catheter and an inner catheter supporting an implant. The system further includes a stabilization device having a distal end fixably connected to the outer catheter and a proximal end movable with respect to the outer catheter. The stabilization device has a delivery arrangement in which the stabilization device is positioned against the outer catheter along a length of the stabilization device and a deployed arrangement in which the proximal end of the stabilization device bulges outwardly from the outer catheter.

In yet another aspect, the disclosure provides a method including the steps of providing a system including a delivery device having an outer catheter and an inner catheter supporting an implant. The delivery device further having a stabilization device positioned at least partially over the outer catheter; the stabilization device including an outer surface and an opening within the outer surface between a distal end and a proximal end of the stabilization device. The method includes delivering the delivery device to an atrium of a heart such that the stabilization device engages an inferior vena cava and directing the implant out of the opening to a valve annulus. Then, the implant is deployed into the valve annulus.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a schematic diagram of a system of the disclosure including a delivery device delivering an implant (not visible) to a tricuspid valve.

FIG. 2A is a side view of the delivery device of FIG. 1.

FIG. 2B is a perspective view of the delivery device of FIGS. 1-2A.

FIG. 3 is a schematic view of the delivery device of FIGS. 1-2B having a stabilization device further including a support.

FIG. 4A is a side view of the stabilization device and support of FIG. 3 in a delivery arrangement.

FIG. 4B is a side view of the stabilization device and support of FIGS. 3-4A in a deployed arrangement.

FIG. 5 is a side view of an alternate stabilization device having two supports in a deployed arrangement.

FIG. 6A is a side view of the stabilization device of FIGS. 4A-4B having an alternate support that is inflatable, the support shown in a deployed arrangement.

FIG. 6B is a cross-sectional view of the support of FIG. 6A in the deployed arrangement.

FIG. 7A is a side view of an alternate system including a delivery device sheathing the implant and having a stabilization device in a delivery arrangement.

FIG. 7B is a side view of the system of FIG. 7A, the stabilization device in a deployed arrangement.

FIG. 8A is a side view of an alternate system including a delivery device sheathing the implant and having a stabilization device in a delivery arrangement.

FIGS. 8B-8C are side views of the system of FIG. 8A, the stabilization device in a deployed arrangement.

FIGS. 9-11 collectively illustrate an alternate system of the disclosure.

FIGS. 12-15 collectively illustrate yet another system of the disclosure.

### DETAILED DESCRIPTION

Specific embodiments of the present disclosure are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

As discussed herein, delivery devices of the disclosure are used to deliver and deploy a cardiac implant at a target site. As referred to herein, implants, stented prostheses, stented prosthetic heart valves or “prosthetic valves” are referenced interchangeably and are useful with the various systems, devices and methods of the present disclosure may assume a wide variety of configurations. Stented prosthetic heart valves can include, for example, a bioprosthetic heart valve having tissue leaflets or a synthetic heart valve having polymeric, metallic or tissue-engineered leaflets, and can be specifically configured for replacing valves of the human heart. The prosthetic valves and stented prostheses of the present disclosure may be self-expandable, balloon expandable and/or mechanically expandable or combinations thereof. In general terms, the prosthetic valves of the present disclosure include a stent or stent frame having an internal lumen maintaining a valve structure (tissue or synthetic), with the stent frame having a normal, expanded condition or arrangement and collapsible to a compressed condition or arrangement for loading within the delivery device. For example, the stents or stent frames are support structures that comprise a number of struts or wire segments arranged relative to each other to provide a desired compressibility and strength to the prosthetic valve. The struts or wire segments are arranged such that they are capable of self-transitioning from, or being forced from, a compressed or collapsed arrangement to a normal, radially expanded arrangement. The struts or wire segments can be formed from a shape memory material, such as a nickel titanium alloy (e.g., nitinol). The stent frame can be laser-cut from a single piece of material, or can be assembled from a number of discrete components. One non-limiting example of an implant suitable for use with the systems of the disclosure can be found in U.S. patent Ser. No. 15/643,011, the disclosure of which is hereby incorporated by reference.

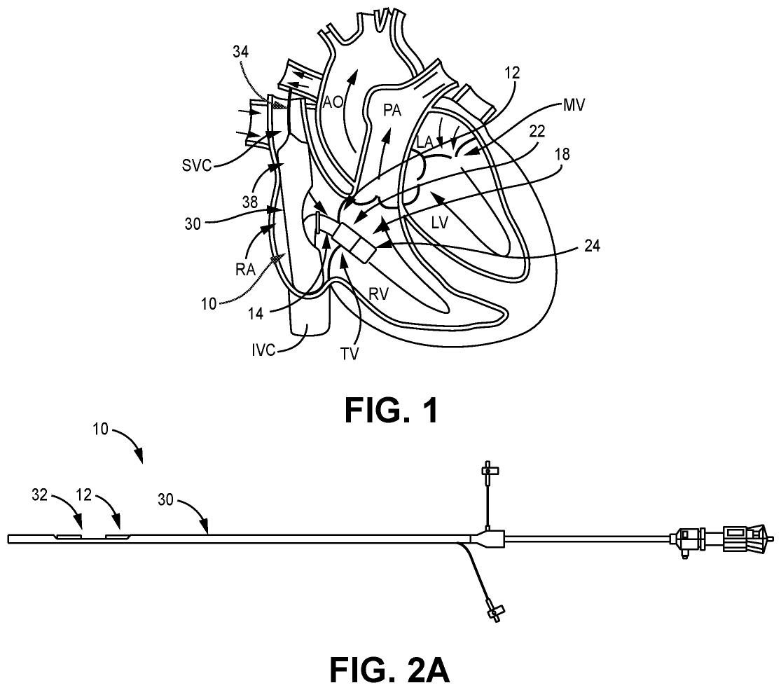

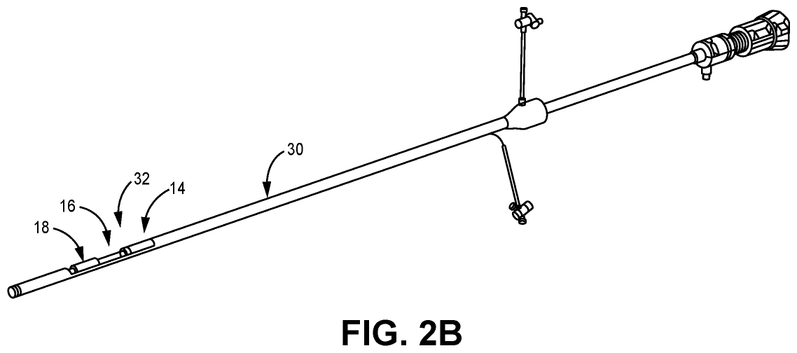

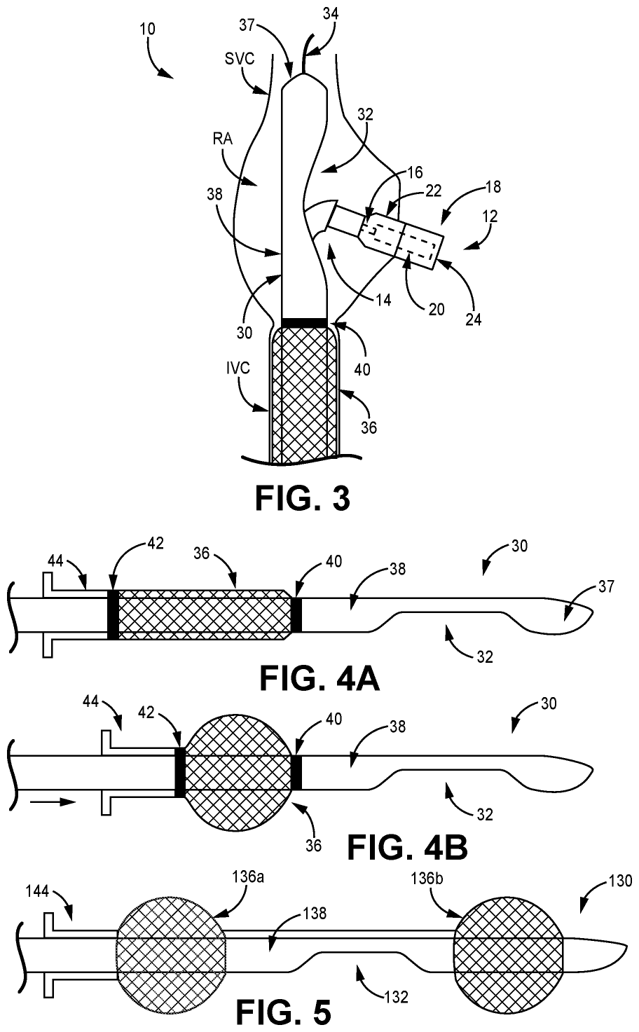

Certain aspects of the present disclosure relate to systems including transcatheter stented prosthetic heart valve delivery devices that retain the implant in a compressed arrangement during delivery to a target site and allow the prosthetic valve to expand and deploy at a target site. Other aspects relate to delivery and deployment methods with such systems and devices. One example of a procedure for treating a patient having a human heart including an inferior vena cava IVC, superior vena cava SVC, right atrium RA, right ventricle RV, tricuspid valve TV, left atrium LA, left ventricle LV, mitral valve MV septal wall, left atrium LA and left ventricle LV is generally depicted in FIG. 1. In this example, a system 10 includes a delivery device 12 having one or more coaxially arranged catheters (see also, FIGS. 2A-2B). The one or more catheters can include an outer catheter 14 and an inner catheter 16. The delivery device 12 further includes a capsule 18 for housing an implant 20 (see also FIG. 3) of any of the type disclosed herein. In the illustrated example, the capsule 18 includes a proximal portion 22 secured to the outer catheter 14 and a distal portion 24 secured to an innermost catheter or shaft movable within the inner catheter 16 on which the implant 20 is supported (see also, FIG. 2B illustrating the inner catheter 16). The present disclosure is not intended to be limited to a capsule having two portions and can optionally include a capsule including a single portion to sheath the implant 20. The capsule 18 can optionally include echogenic markers (not shown) to aid with positioning. Other delivery devices suitable for delivering and deploying the implant via transcatheter procedure are suitable for use with systems of the disclosure.

In the example method of tricuspid repair shown in FIG. 1, the delivery device 12 is maneuvered, typically routed through the inferior vena cava IVC to the right atrium RA and continues to the tricuspid valve TV. In another example, the delivery path can continue to the patient's septal wall, through an aperture formed in the patient's septal wall, to the left atrium LA and resume by turning approximately 90° downward through the native mitral valve MV. For either procedure, one of the difficulties in positioning the implant 20 is stabilizing the system 10 during the procedure, particularly as the delivery device 12 is making turns. For a mitral valve MV replacement procedure, although the point at which the outer catheter 14 passes through the septal wall acts in some way as a stabilization point for the delivery device 12, heart tissue can be damaged during the articulation of the capsule 18 into the left atrium LA. Additionally, the outer catheter 14 almost inevitably pushes against the side of the inferior vena cava IVC, just inferior to the right atrium RA (assuming trans-femoral venous access), which can cause damage to the inferior vena cava IVC and/or perhaps, other heart tissue. For a tricuspid valve TV procedure, any potential support from the septal wall is not available. It is believed that the embodiments of the present disclosure will allow for atraumatic stabilization of a delivery device in one or both of the superior vena cava and inferior vena cava causing less trauma to the inter atrial septum and the right or left atrium as the capsule is articulated in the respective atrium. The disclosed embodiments are believed to further improve capsule control for the physician, which is further believed to reduce the opportunity for damage to the inter-atrial septum and/or other tissue of the patient.

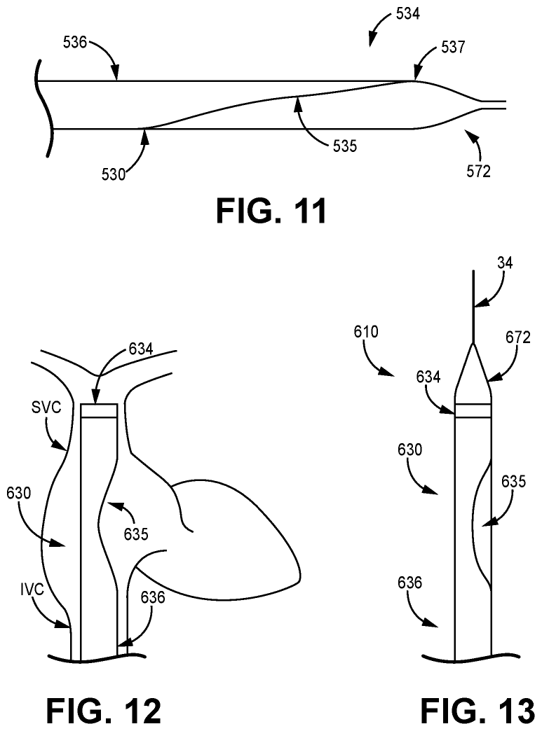

Referring now also to FIGS. 2A-4B, which illustrate select components of the system 10 including a stabilization device 30 coaxially positioned around at least a portion of the outer catheter 14 of the delivery device 12 during delivery. Optionally, the stabilization device 30 can have a tapered or pointed tip 37, which can optionally be configured similar to a dilator (see also, FIGS. 11 and 13, and related disclosure). In various examples, the tip 37 is closed. The stabilization device 30 is configured such that during deployment, the stabilization device 30 can be positioned within both of the inferior vena cava IVC and the superior vena cava SVC with an opening 32 of the stabilization device 30 being adjacent the tricuspid valve annulus, for example. In some examples, the stabilization device 30 is delivered over a guide wire 34. In some examples, the guide wire 34 is anchored to a subclavian vein. In some optional embodiments, the stabilization device 30 can further include a support 36. In this example, the support 36 is made of expandable mesh or the like. In various examples, the mesh is made of a shape memory or otherwise biased material (e.g., nitinol) so that the support 36 can be delivered in a compressed arrangement, compressed by a sheath, catheter or the like (see also, sheath 368 of FIG. 7A and related disclosure) and released and freed from the confines of the guide catheter by proximally withdrawing the guide catheter to naturally expand the support 36 to its expanded arrangement. In the expanded arrangement, the support 36 is non-occluding and has a greater outer diameter as compared to the delivery arrangement so that the support 36 can engage the adjacent anatomy and support the delivery device 12. The stabilization device 30 is configured to have the side opening 32 formed in its outer surface 38. In this way, the capsule 18 can exit the side opening 32 at an angle with respect to a central axis of the stabilization device 30.

Referring now in particular to FIGS. 3-4B, in one example, the support 36 is made of metal mesh or the like and has a first ring 40 and a second ring 42. The mesh may made of braided shape memory meatal or spring steel, for example. The first ring 40 is fixably secured to the outer surface 38 of the stabilization device 30, proximal to the opening 32, and the second ring 42 is slidably secured around the outer surface 38. The second ring 42 is secured to an actuation shaft 44 (which is shown as shortened for ease of illustration). To transition the support 36 from the delivery arrangement (FIG. 4A) to the expanded arrangement (FIG. 4B), the actuation shaft 44 is distally advanced to force the support 36 to bulge outwardly with respect to the outer surface 38. In some embodiments, the rings 40, 42 can be omitted and the support 36 can be directly connected to the outer surface 38 and/or actuation shaft 44.

Referring now in addition to FIG. 5, which illustrates an alternate stabilization device 130 that can be used with any of the devices and systems of the disclosure. In this example, the stabilization device 130 includes two supports 136a, 136b on opposing sides of an opening 132 in an outer surface 138 of the stabilization device 130. In one example, one support 136a is positioned within the inferior vena cava and the second support 136b is positioned in the superior vena cava during delivery of an implant (e.g., implant 20). Each support 136a, 136b can be made of any material disclosed herein for alternate supports and can otherwise be configured identically to brace the delivery device 12 during delivery. Further, each support 136a, 136b can be transitioned from the delivery arrangement to the deployed arrangement in a variety of manners. In one example, each distal ring or distal end of the support 136a, 136b can be secured to the outer surface 138 and each proximal ring or proximal end of the support 136a, 136b can be distally advanced with its own or a similar actuation shaft 144 (shown as truncated for ease of illustration) to allow the supports 136a, 136b to budge outward with respect to the outer surface 138 in the deployed arrangement.

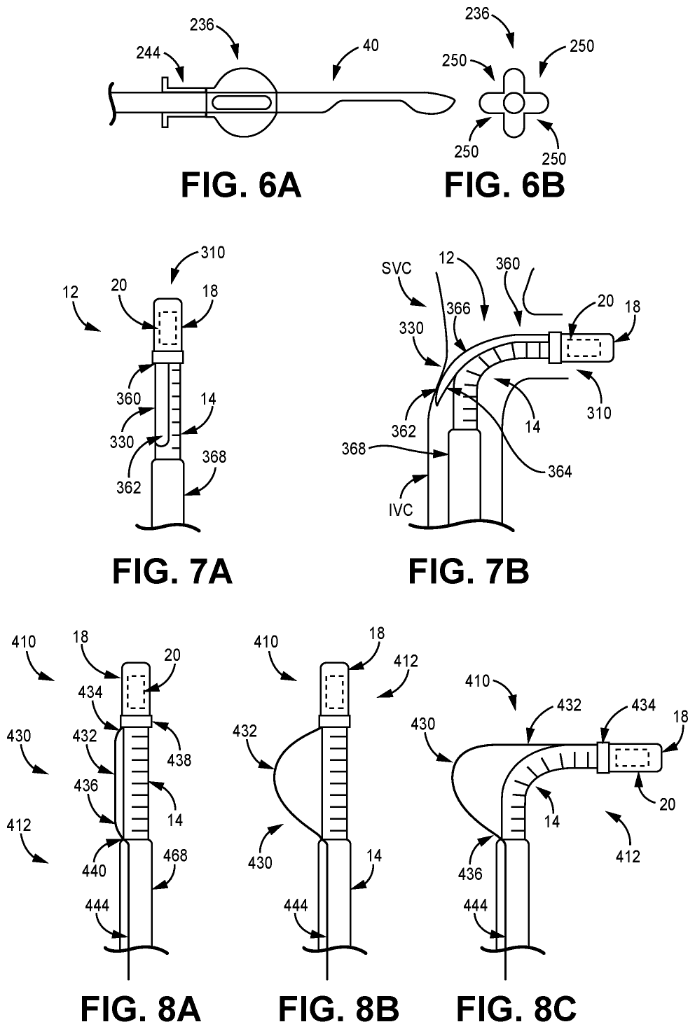

Referring now in addition to FIGS. 6A-6B, in some examples, one or more supports of the delivery devices of the disclosure is configured to be an inflatable balloon 236 having a delivery arrangement in which the balloon 236 is deflated and a deployed arrangement in which the balloon 236 is inflated. Optionally, the balloon 236 can be made of nylon, urethane or other compliant material. In some embodiments, the balloon 236 is configured to be non-occluding in the deployed arrangement shown in FIGS. 6A-6B. In the present example, the balloon 236 inflates to define a plurality of channels 250 (e.g., four channels extending along a length of the balloon 236) through which blood can flow while the balloon 236 is in the deployed arrangement and engaged with the anatomy. Optionally, the balloon 236 can be at least partially supported on shaft 244 positioned over the stabilization device 30. An inflation path (not referenced) may be provided in the shaft 244 and may terminate at the balloon 236.

Referring now in addition to FIGS. 7A-7B, which illustrate a system 310 including the delivery device 12 supporting implant 20 as well as an alternate stabilization device 330. In this embodiment, the stabilization device 330 has a distal end 360 fixedly secured to the outer catheter 14, adjacent the capsule 18. In one example, the stabilization device 330 is made of a polymer material such as nylon or polyether block amide. A proximal end 362 of the stabilization device 330 is not secured to the outer catheter 14 such that the stabilization device 330 can transition from a delivery arrangement of FIG. 7A in which an entire length of the stabilization device 330 is contacting or against the outer catheter 14 to a deployed arrangement in which the outer catheter 14 is bent such that the proximal end 362 of the stabilization device 330 extends away from and is spaced from the outer catheter 14. The stabilization device 330 can include curved inner and outer surfaces 364, 366 to conform to the outer catheter 14 and to fit within the sheath 368 that can be selectively positioned over the delivery device 12 and provided as part of the system 310.

In one example, the system 310 is directed through the inferior vena cava IVC with the sheath 368 covering the stabilization device 330. The sheath 368 is proximally withdrawn and the delivery device 12 is and directed to the right atrium RA. The bend in the outer catheter 14 causes the proximal end 362 of the stabilization device 330 to extend away from the outer catheter 14 and engage the inferior vena cava IVC, supporting and bracing the delivery device 12 as the inner catheter 16 within outer catheter 14, which supports capsule 18, is directed through the tricuspid valve annulus TV and into the right ventricle RV to deliver and deploy the implant 20 at the tricuspid valve TV (see also, FIG. 1). After the implant 20 is deployed at the tricuspid valve TV, the delivery device 12 can be withdrawn proximally through the deployed implant 20 until the stabilization device 330 is generally linear and the proximal end 362 is against the outer catheter 14. Then, the sheath 368 can be distally advanced over the stabilization device 330 and the system (minus the deployed implant) 310 can be withdrawn in the same manner as delivered.

Referring now in addition to FIGS. 8A-8C, which illustrate a system 410 including a delivery device 412 supporting the implant 20 as well as having an alternate stabilization device 430. In this example, the stabilization device 430 includes a flexible interconnecting member 432 having a distal end 434 and a proximal end 436. Optionally, the interconnecting member 432 can be made of a flat sheet or round wire material being a polymer, shape memory alloy or spring steel. The distal end 434 is fixedly secured to the outer catheter 14 adjacent the capsule 18, optionally with a ring 438, and the proximal end is slidably secured to the outer catheter 14. In one embodiment, the proximal end 436 is secured to a ring 440 that is positioned around the outer catheter 14. The proximal end 436 or ring 440 can be secured to an actuation shaft or the like 444. In a delivery arrangement (FIG. 8A), the actuation shaft 444 is tensioned to align the interconnecting member against the outer catheter 14 along a length of the outer catheter 14. When the outer catheter 14 is to be articulated during delivery, slack can be provided in the interconnecting member 432 by releasing tension in the actuation shaft 444 to allow the interconnecting member to bow outwardly with respect to the outer catheter 14 (FIG. 8B) to allow the outer catheter 14 room and freedom to bend in the direction of the interconnecting member 432 (FIG. 8C). In one method, the delivery device 412 is directed through the inferior vena cava in the delivery arrangement. Optionally, the stabilization device 430 can be delivered sheathed by a sheath, catheter or the like 468. In various embodiments, the actuation shaft 444 can be at least partially routed through the outer catheter 14 or guide catheter 468. When the capsule 18 reaches the right atrium, the interconnecting member 432 is allowed to bow outwardly (FIG. 8B) and then the capsule 18 is angled into the right atrium and directed to the tricuspid annulus for replacement of a tricuspid valve with the implant 20. As the interconnecting member 432 bows outwardly, the interconnecting member 432 is configured to abut the atrial wall or vena cava and support the outer catheter 14 as the outer catheter 14 bends to position the inner catheter 16 (visible in FIGS. 2B and 3) and capsule 18 to deploy the implant 20 within the capsule 18.

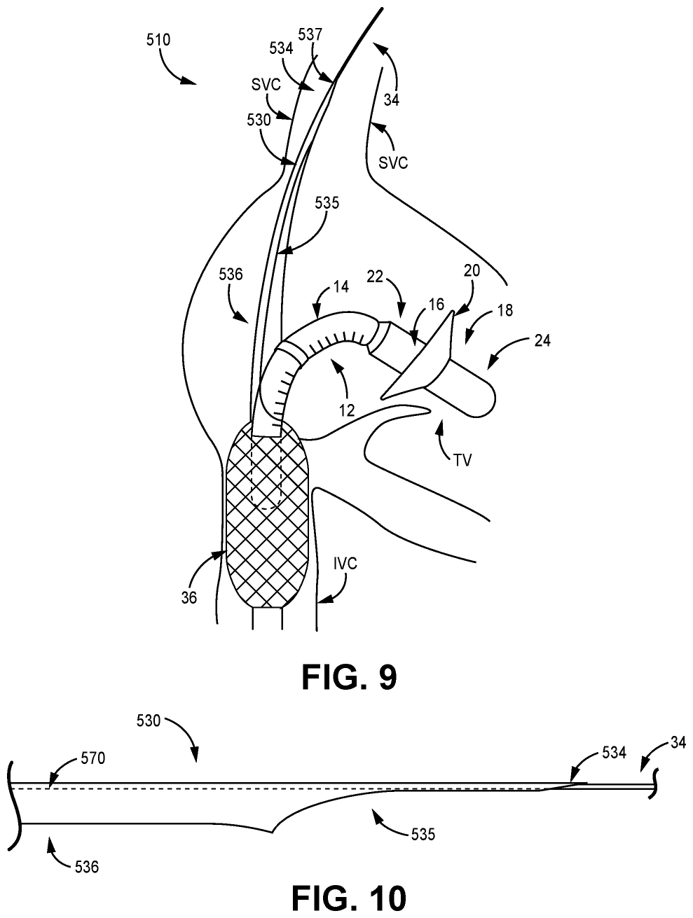

Referring now in addition to FIGS. 9-10, which illustrate an additional system 510 of the disclosure. The system 510 includes the delivery device 12 supporting the implant 20 as discussed above with respect to the embodiment of FIG. 1, however, alternate delivery devices can also be used with the system 510. In this example, the delivery device 12 is optionally supported within the inferior vena cava IVC with a stabilization device 530 that can optionally include support 36. Alternate inferior vena cava IVC support can be provided as disclosed herein and utilized with system 510. The stabilization device 530 includes a tapered distal end 534 including an opening 535 that extends from a proximal end 536 of the stabilization device 530 to a distal tip 537, which may be pointed. In various examples, the system 510 can include the guide wire 34, which can extend through the stabilization device 530 and out of the opening 535 at the tip 537. In various examples, the guide wire 34 is anchored to the superior vena cava SVC. In one example, the guide wire 34 routed through a lumen 570 formed separately from opening 535. In various embodiments, the system 510 can be provided with a dilator 572 as shown in FIG. 11. The dilator 572 be inserted through the stabilization device 530 and extend at least partially out of the opening 535 and past the tip 537.

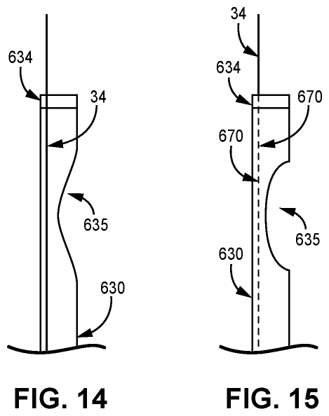

Referring now in addition to FIGS. 12-14, which collectively illustrate an additional system 610 of the disclosure. The system 610 can include the delivery device 12 (not shown) supporting the implant 20 as discussed above with respect to the embodiment of FIG. 1, however, alternate delivery devices can also be used with the system 610 in a manner similar to system 510. In this example, a delivery device includes a stabilization device 630 configured to be simultaneously positioned in the inferior vena cava IVC and the superior vena cava SVC. Alternate inferior vena cava IVC support can be provided as disclosed herein and utilized with system 610. The stabilization device 630 includes an open distal end 634 and a side opening 635 within a side of the stabilization device 630 between the distal end 634 and a proximal end 636 of the stabilization device 630. In various examples, the system 610 can include the guide wire 34, which can extend through the stabilization device 630 and out of the open distal end 634. The guide wire 34 can be routed externally from the stabilization device 630 (FIG. 14) or within a lumen 670 formed within the stabilization device 630, separate from the opening 635 (FIG. 15). In various examples, the guide wire 34 is anchored to the superior vena cava SVC. In various embodiments, the system 610 can be provided with a dilator 572 as shown in FIG. 13. The dilator 672 be inserted through the stabilization device 630 and extend at least partially out of the open distal end 634 and past the stabilization device 630. In some examples, the guide wire 34 is routed through the dilator 672. Except as expressly stated, system 610 can be identically configured and used in a similar manner as systems 510 or 10.

In various example methods of the disclosure, a method includes the steps of providing a system of the disclosure including a delivery device having an outer catheter and an inner catheter supporting an implant. The delivery device further has a stabilization device positioned at least partially over the outer catheter. The stabilization device includes an outer surface and an opening within the outer surface between a distal end and a proximal end of the stabilization device. Methods can include delivering the delivery device to an atrium of a heart such that the stabilization device engages an inferior vena cava and directing the implant out of the opening to a valve annulus. Then, the implant is deployed into the valve annulus. In various methods, during the step of delivering the delivery device, the stabilization device further engages a superior vena cava. In some examples, the stabilization device includes a first support on the outer surface, the first support having a delivery arrangement and an expanded arrangement. A maximum outer diameter of the first support can be greater in the expanded arrangement as compared to the delivery arrangement. In some methods, the first support is transitioned to the delivery arrangement at the inferior vena cava. In various examples, the first support is an inflatable balloon. Alternatively, the first support is made of mesh. Optionally, the first support is biased to the expanded arrangement. In some examples, the first support is connected to an actuation shaft configured to transition the first support from the delivery arrangement to the expanded arrangement. In some examples, the first support is positioned proximal to the opening. In various embodiments, a second support is positioned on the outer surface distal to the opening. The second support can have a compressed arrangement and an expanded arrangement. The second support can have a greater outer diameter in the expanded arrangement as compared to the compressed arrangement. In various methods, the stabilization device receives a guide wire that is optionally anchored to a subclavian vein. Various methods include a capsule sheathing the valve during the step of delivering the implant. The capsule can optionally include a distal portion and a proximal portion, wherein the distal portion and the proximal portion are configured to separate to release or unsheathe the implant. In various methods, the distal end of the stabilization device is secured to the outer catheter and the proximal end of the stabilization device extends away from the outer catheter to engage the inferior vena cava. In various methods, one of the distal end and proximal ends of the stabilization device is fixed to the outer catheter and another of the distal and proximal ends is movable with respect to the distal end. The stabilization device can be configured to transition from a delivery arrangement in which the stabilization device is positioned against the outer catheter to a deployed arrangement in which the stabilization device bulges outwardly with respect to the outer catheter to engage the inferior vena cava.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A system comprising:
a delivery device having an outer catheter and an inner catheter supporting an implant; and
a stabilization device positioned at least partially over the outer catheter, the stabilization device including a distal end and a proximal end; the stabilization device including an outer surface and an opening within the outer surface positioned between the distal end and the proximal end;
wherein the system includes an arrangement in which the outer catheter at least partially extends out of the opening.

2. The system of claim 1, wherein the stabilization device includes a first support on the outer surface, the first support having a delivery arrangement and an expanded arrangement; further wherein a maximum outer diameter of the first support is greater in the expanded arrangement as compared to the delivery arrangement.

3. The system of claim 2, wherein the first support is an inflatable balloon.

4. The system of claim 2, wherein the first support is made of mesh.

5. The system of claim 2, wherein the first support is biased to the expanded arrangement.

6. The system of claim 2, wherein the first support is connected to an actuation shaft configured to transition the first support from the delivery arrangement to the expanded arrangement.

7. The system of claim 2, wherein the first support is positioned proximal to the opening.

8. The system of claim 2, further comprising a second support positioned on the outer surface distal to the opening; wherein the second support has a compressed arrangement and an expanded arrangement; wherein the second support has a greater outer diameter in the expanded arrangement as compared to the compressed arrangement.

9. The system of claim 1, wherein a capsule is positioned to sheath the implant.

10. The system of claim 9, wherein the capsule includes a distal portion and a proximal portion, wherein the distal portion and the proximal portion are configured to separate to release the implant.

11. A system comprising:
a delivery device having an outer catheter and an inner catheter supporting an implant; and
a stabilization device having a distal end fixably connected to the outer catheter and a proximal end movable with respect to the outer catheter;
wherein the stabilization device has a delivery arrangement in which the stabilization device is positioned against the outer catheter along a length of the stabilization device and a deployed arrangement in which the proximal end of the stabilization device bulges outwardly from the outer catheter.

12. The system of claim 11, wherein the proximal end is connected to an actuation shaft configured to transition the stabilization device form the delivery arrangement to the deployed arrangement.

13. The system of claim 11, wherein the implant is a prosthetic heart valve.

14. The system of claim 11, wherein the distal end is adjacent a capsule sheathing the implant.

15. The system of claim 11, wherein the stabilization device includes an outer surface and an inner surface; wherein the inner and outer surfaces are curved.

16. The system of claim 11, wherein the proximal end is slidingly secured to the outer catheter.

17. A method comprising:
providing a system including a delivery device having an outer catheter and an inner catheter supporting an implant, the delivery device further having a stabilization device positioned at least partially over the outer catheter; the stabilization device including an outer surface and an opening within the outer surface between a distal end and a proximal end of the stabilization device;
delivering the delivery device to an atrium of a heart such that the stabilization device engages an inferior vena cava;
directing the implant out of the opening to a valve annulus; and
deploying the implant into the valve annulus.

18. The method of claim 17, wherein the stabilization device includes a first support on the outer surface, the first support having a delivery arrangement and an expanded arrangement; further wherein a maximum outer diameter of the first support is greater in the expanded arrangement as compared to the delivery arrangement; further wherein the first support is transitioned to the delivery arrangement at the inferior vena cava.

19. The method of claim 17, wherein the distal end is secured to the outer catheter and the proximal end extends away from the outer catheter to engage the inferior vena cava.

20. The method of claim 17, one of the distal end and proximal ends of the stabilization device is fixed to the outer catheter and another of the distal and proximal ends is movable with respect to the distal end; the stabilization device configured to transition from a delivery arrangement in which the stabilization device is positioned against the outer catheter to a deployed arrangement in which the stabilization device bulges outwardly with respect to the outer catheter to engage the inferior vena cava.
